(19) United States
(12) Patent Application Publication
(10) Pub. No.: US 2019/0266001 A1
(43) Pub. Date: Aug. 29, 2019

(54) APPLICATION PROVIDING APPARATUS, APPLICATION PROVIDING METHOD, AND APPLICATION PROVIDING PROGRAM

(71) Applicant: FUJIFILM Corporation, Tokyo (JP)

(72) Inventor: Erimu HARADA, Tokyo (JP)

(73) Assignee: FUJIFILM Corporation, Tokyo (JP)

(21) Appl. No.: 16/284,660

(22) Filed: Feb. 25, 2019

(30) Foreign Application Priority Data
Feb. 28, 2018 (JP) 2018-035194

(51) Int. Cl. G06F 9/455; H04L 29/08; G16H 30/40; H04L 12/24

(52) U.S. Cl. CPC G06F 9/45558; H04L 67/327; G06F 2009/4557; H04L 67/34; H04L 41/0806; G16H 30/40

(57) ABSTRACT

There is performed a setting of a common virtual machine to which a parameter for any user of a plurality of users is set, as a dedicated virtual machine of a user for which a setting of the parameter is performed, in the common application program set in each of a plurality of common virtual machines, and there is provided a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.

### CROSS-REFERENCE TO RELATED APPLICATION

This application claims priority from Japanese Patent Application No.2018-035194, filed on Feb. 28, 2018, the disclosure of which is incorporated by reference herein in its entirety.

### BACKGROUND OF THE INVENTION

### Field of the Invention

The present invention relates to an application providing apparatus, an application providing method, and an application providing program.

### Related Art

In recent years, since various kinds of cloud services are provided, application software (hereinafter, referred to as an application in short) has been provided using a cloud without possessing expensive computers as well as data have been preserved using the cloud. By using such an Application Service Provider (ASP) service to leave maintenance, operation, and management of hardware to a company providing the cloud, it is intended to reduce costs and human resources.

In a medical field, a diagnosis performance is improved due to a progress in an image processing technology. In recent years, a recognition accuracy of lesions is particularly improved by using deep learning using neural networks. Computers having a high performance are required for using such a new technology. Also, different functions are used depending on hospitals, and it is needed to perform different settings accordingly.

At present, an application may correspond to computers of a plurality of makers, but in a case where the application is provided to a user, it is needed to perform different environmental settings depending on a maker of a computer owned by the user, the number of computers, and a kind of a usable network. In JP2013-131134A, an application included in a service is specified and a profile of a user terminal is extracted, and then based on a setting template of the specified application, an original setting file applicable to the user terminal is automatically generated.

Also in the medical field, it is considered to provide an application used in diagnosis by using the ASP service. However, different settings are required according to different functions used for each hospital, or setting for allocation of resources (a CPU, a memory, a hard disk, a network, and the like) and the number of accounts to be required according to a utilization rate are needed to be performed. One merit for use of the ASP service is that it is possible to use a new application without possessing expensive computers. However, since expenses are generated corresponding to provided services, the merit for use of the ASP service diminishes, in a case where the number of accounts increases more than necessary or allocation of resources increases more than necessary. Also, since data used in the medical field is personal information, the provided services are expensive in the view of security protection.

### SUMMARY

In order to address above-mentioned problem, an object of the present invention is to provide an application providing apparatus, an application providing method, and an application providing program for providing a cloud structure which makes it possible to secure proper resources and provide execution environments of applications.

According to an embodiment of the present invention, there is provided an application providing apparatus comprising: virtual machine starting means for starting each of a plurality of common virtual machines in which a common application program is set; dedicated virtual machine setting means for setting the common virtual machine to which a parameter for any user of a plurality of users is set, as a dedicated virtual machine of the user for which a setting of the parameter is performed, in the common application program set in each of the plurality of common virtual machines; and function providing means for providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.

According to another embodiment of the present invention, there is an application providing method, comprising: a virtual machine starting step of starting each of a plurality of common virtual machines in which a common application program is set; a dedicated virtual machine setting step of setting the common virtual machine to which a parameter for any user of a plurality of users is set, as a dedicated virtual machine of the user for which a setting of the parameter is performed, in the common application program set in each of the plurality of common virtual machines; and a function providing step of providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient, which are performed by an application providing apparatus.

According to still another embodiment of the present invention, there is provided an application providing program that causes a computer to perform: a virtual machine starting step of starting each of a plurality of common virtual machines in which a common application program is set; a dedicated virtual machine setting step of setting the common virtual machine to which a parameter for any user of a plurality of users is set, as a dedicated virtual machine of the user for which a setting of the parameter is performed, in the common application program set in each of the plurality of common virtual machines; and a function providing step of providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.

“Resources” mean physical resources of a computer such as a CPU, a memory, and a storage of a computer, and a network.

Also, in a case where the utilization rate of resources of a plurality of dedicated virtual machines used by one user is equal to or less than a reference value, it is desirable that the function providing means stops use of at least one of the plurality of dedicated virtual machines of the one user.

Also, each of a plurality of common virtual machines may be a virtual machine to which a constant resource is allocated.

Also, in a case where each of the plurality of users is a hospital, the common application program includes a program for performing image processing or a program for performing diagnosis report creation support processing, in which the setting of the parameter is a setting of an image processing parameter of the image processing corresponding to the hospital in a case of the program for performing the image processing, and a setting of a format of a diagnosis report of the diagnosis report creation support processing corresponding to the hospital in a case of the program for performing the diagnosis report creation support processing.

Also, each of the plurality of common virtual machines has a storage unit in which data used for the common application program is readable or writable, and the setting of the parameter allows the storage unit to be set as a dedicated storage unit with respect to a user for which the setting of the parameter is performed.

The application providing apparatus of the present invention performs processing for starting each of a plurality of common virtual machines in which a common application program is set; setting the common virtual machine to which a parameter for any user of a plurality of users is set, as a dedicated virtual machine of the user for which a setting of the parameter is performed, in the common application program set in each of a plurality of common virtual machines; and providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.

According to embodiments of the invention, it is possible to provide the cloud structure which makes it possible to secure proper resources and provide execution environment of applications.

### BRIEF DESCRIPTION OF THE DRAWINGS

FIG. 1 is a diagram showing one example of a schematic configuration of an application providing system.

FIG. 2 is a schematic configuration diagram showing one example of an application providing apparatus of the present embodiment.

FIG. 3 is a diagram for explaining a change from a common virtual machine to a dedicated virtual machine.

FIG. 4 is one example of a flowchart showing a flow of processing for starting a common virtual machine.

FIG. 5 is one example of a flowchart showing a flow of processing for changing a started common virtual machine into a dedicated virtual machine.

### DETAILED DESCRIPTION

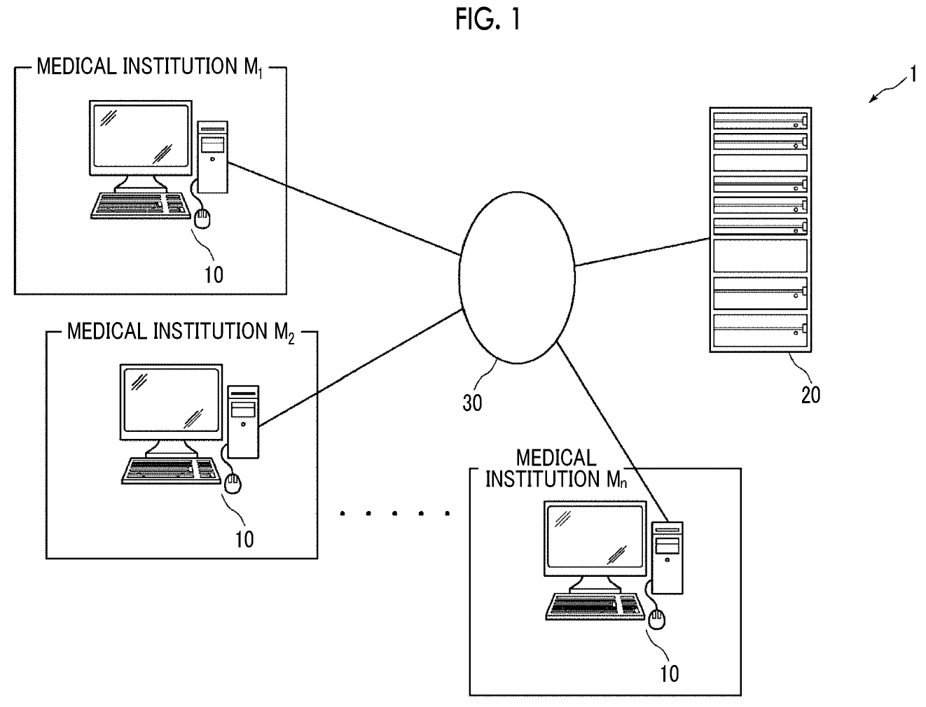

FIG. 1 shows a schematic configuration of an application providing system 1 in a first embodiment of the present invention. In the present embodiment, there is described a case where an application for image processing is provided to a medical institution. The application providing system 1 is configured such that a plurality of terminal apparatuses 10, which are provided in a plurality of medical institutions M₁, M₂, and, . . . , Mₙ, and an application providing apparatus 20, which is placed in a cloud side (hereinafter, referred to as an application providing server 20), are connected with one another in a network 30.

The application providing server 20 is a computer having a well-known hardware configuration such as a Central Processing Unit (CPU), a memory, a storage, an input/output interface, a communication interface, and a data bus. Also, a Graphics Processing Unit (GPU) may be provided as necessary. The application providing program of an embodiment of the present invention is installed on the computer, and functions as the application providing server 20 through the execution of the commends of the program by the CPU. Also, a hypervisor, and the like may be installed on the application providing server 20.

The application providing program is stored in a network storage or a storage apparatus of a server computer connected to a network in a state of being accessible from the outside. In response to a request from the outside, the application providing program is downloaded and installed into the computer. Alternatively, the application providing program may be recorded in a recording medium such as a Digital Versatile Disc (DVD) and a Compact Disc Read Only Memory (CD-ROM) to be distributed and may be installed from the recording medium into the computer.

A plurality of virtual machines are installed on the hypervisor, an executable operating system (hereinafter, referred to as an OS) is mounted on each virtual machine, and the application is installed on the mounted OS. Also, the hypervisor has a function for managing support for an I/O device, which is usable by each virtual machine, memory management, or management of an external storage apparatus and the like.

The hypervisor may operate directly on hardware, or in a way that all the OSs operate on the virtual machines of the hypervisor. Alternatively, the hypervisor may operate in a way that at first, a host OS operates on hardware, and on the host OS, the hypervisor operates as an application of the host OS, and furthermore, other guest OSs operate on the virtual machines which operate on the hypervisor.

A terminal apparatus 10 is a computer provided in each medical institution M₁, M₂, and, . . . , Mₙ, and has a well-known hardware configuration of a CPU, a memory, a storage, an input/output interface, a communication interface, an input apparatus, a display apparatus, and a data bus and the like, and includes a display as the display apparatus. Also, a well-known operation system, and the like are installed on the terminal apparatus 10.

Also, the terminal apparatus 10 is connected to a medical information system of each medical institution M₁, M₂, and, . . . , Mₙ, through a Local Area Network (LAN) (not shown). Also, the medical information system comprises an image database for storing image data of patients which are imaged by various kinds of modalities and a diagnosis report database for storing diagnosis reports on which diagnosis results are recorded. For example, the modalities include a Computed Tomography (CT) apparatus, a Magnetic Resonance Imaging (MRI) apparatus, a Positron Emission Tomography (PET) apparatus, an ultrasound apparatus, or a Computed Radiography (CR) apparatus using a Flat Panel Detector (FPD), or the like. Hereinafter, the medical institution is explained as a hospital.

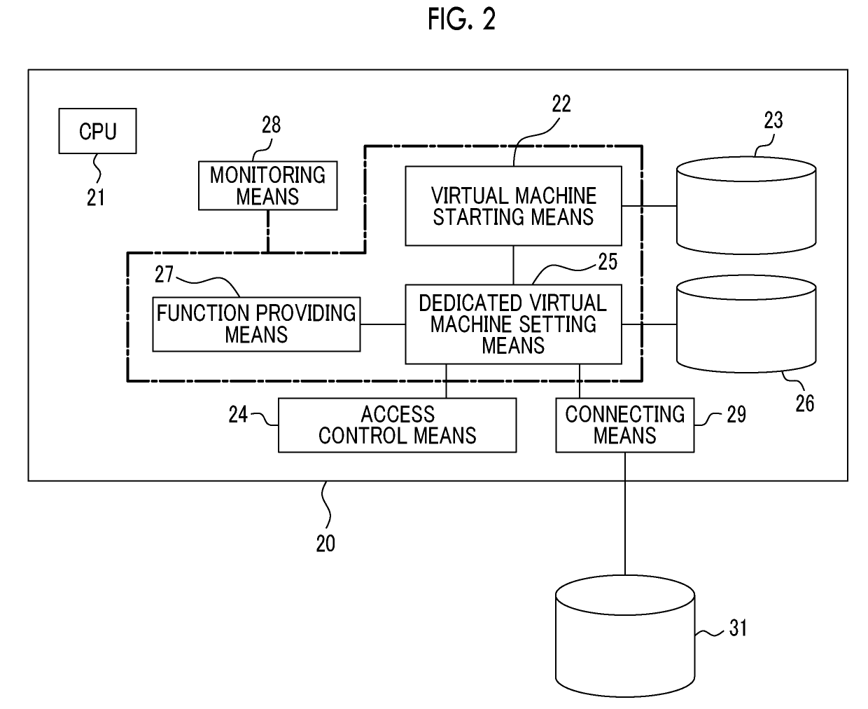

Next, functions of the application providing server 20 will be described. As shown in FIG. 2, the application providing program is executed by a CPU 21, and accordingly functions as virtual machine starting means 22, application storage means 23, access control means 24, dedicated virtual machine setting means 25, parameter storage means 26, function providing means 27, monitoring means 28, and connecting means 29.

The virtual machine starting means 22 starts each of a plurality of common virtual machines in which common applications are set respectively. The common virtual machines are a machine that an OS such as MS Windows (registered trademark) or Unix (registered trademark) is installed on a virtual machine, and further the common application is installed on the OS of the virtual machine. The common application is an application required for providing same functions to a plurality of users, and basic applications required for providing a specified function (for example, image processing) are all installed on the common virtual machine. Also, all of the common virtual machines are set such that a constant resource is allocated. The resource includes, for example, CPU usage, a storage capacity, memory capacity, a network bandwidth, or the number of accesses which may be concurrently accessed.

The application storage means 23 stores a program of the common application used by a plurality of users. The common application is a program for executing a standard function, such as an image processing program. Also, in the case where the user is a hospital, the common application includes a program for performing image processing or a program for performing the diagnosis report creation support processing, and the like.

The dedicated virtual machine setting means 25 sets a common virtual machine to which a parameter for any user of a plurality of users is set, as a dedicated virtual machine of the user for which the setting of the parameter is performed, in the common application program set in each of common virtual machines.

Alternatively, in the case where there are concurrently provided accesses by a plurality of users from one hospital such as hospital M₁, M₂, and, . . . , Mₙ, a dedicated parameter is prepared for each user group which consists of a plurality of users, and by setting that parameter to the common virtual machine, the common virtual machine may be changed into a dedicated virtual machine of the user group.

The setting of the parameter is a setting of an image processing parameter of the image processing corresponding to the hospital in the case of a program for performing the image processing, and a setting of a format of a diagnosis report of the diagnosis report creation support processing corresponding to the hospital in the case of a program for performing the diagnosis report creation support processing.

The parameter storage means 26 stores a parameter to be set in the common application for each user (or each user group). The parameter is prepared as a dedicated parameter corresponding to an identification (ID) of identity information of a user input upon access, and stored in the parameter storage means 26 in association with the ID.

Also, the application storage means 23 and the parameter storage means 26 are provided in a storage of the application providing server 20. Alternatively, the application storage means 23 and the parameter storage means 26 may be provided in a Network Attached Storage (NAS) connected to a network or a disk apparatus connected to Storage Area Network (SAN).

The access control means 24 accepts input of an ID which is input when a user logs in, from the terminal apparatus 10, and authorizes the ID, and in a case where the ID is an ID of an authorized user, the access control means 24 controls a dedicated virtual machine of the user to be accessible. Also, once the user logs out, all the dedicated virtual machines used by the user are released. Alternatively, once all the users belonging to a user group log out, all the dedicated virtual machines are released.

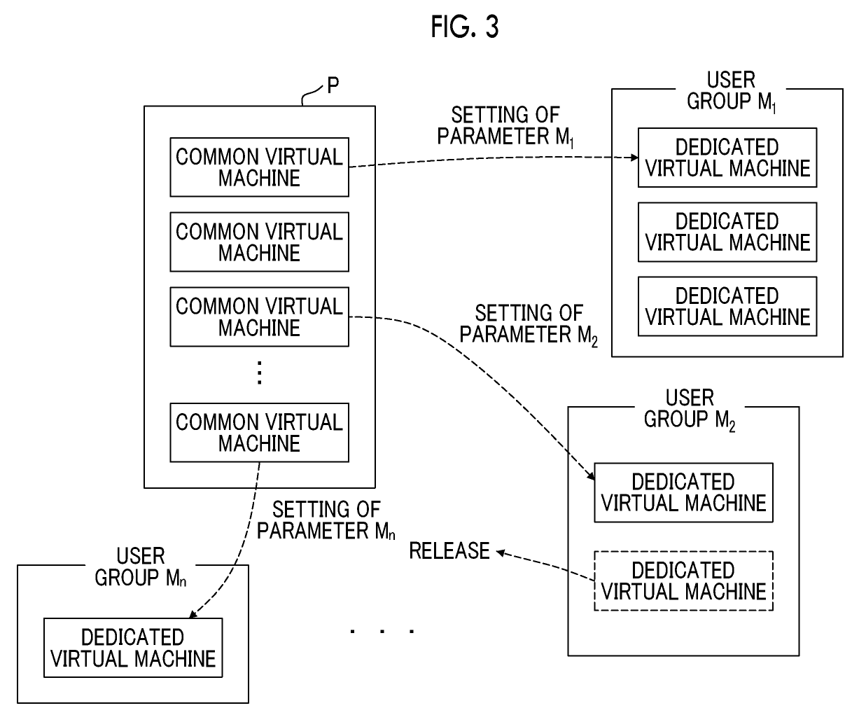

FIG. 3 shows relationships of common virtual machines, dedicated virtual machines, and dedicated parameters in a case where a user group is allocated to the hospital shown in FIG. 1. A plurality of common virtual machines started by the virtual machine starting means 22 are accumulated in a common virtual machine pool P. Once a parameter M₁ for a user group M₁ is set to one of accumulated common virtual machines, this common virtual machine is changed into a dedicated virtual machine of the user group M₁, which may be accessed using an ID of the user group M₁. Similarly, once a parameter M₂ for a user group M₂ is set to another one of accumulated common virtual machines, this common virtual machine is changed into a dedicated virtual machine which may be accessed using an ID of the user group M₂, and once a parameter Mₙ for a user group Mₙ is set to still another one of accumulated common virtual machines, this common virtual machine is changed into a dedicated virtual machine which may be accessed by an ID of the user group Mₙ.

In a case where resources of the dedicated virtual machine are insufficient, the function providing means 27 provides a function by additionally setting at least one of common virtual machines for which a setting of a parameter is not performed, as another dedicated virtual machine of a user corresponding to the dedicated virtual machine of which resources are insufficient. In the case where the resources of the dedicated virtual machine of one user of a plurality of users are insufficient, the dedicated virtual machine setting means 25 adds one of the common virtual machines as a dedicated virtual machine of the user, and functions by the application are provided to one user (or user group). For example, the resources of the dedicated virtual machine are periodically measured, and in the case where the utilization rate of resources is equal to or more than a first reference value, it is determined that the resources are insufficient. In the case where the utilization rate of CPU usage allocated to a dedicated virtual machine used by a specified user exceeds, for example, 80%, once another process except the currently executed process is executed, there is a high possibility not to maintain the processing speed such that the user feels good. For example, it is determined whether or not resources are insufficient, by using the utilization rate of CPU of 80% as the first reference value, and since the resources are insufficient in a case where the utilization rate of CPU exceeds 80%, a dedicated virtual machine is added. In the example of FIG. 3, the user group Mₙ uses just one dedicated virtual machine, but the user group M₁ uses three dedicated virtual machines that have been added. Also, the user group M₂ uses two dedicated virtual machines. Since, in the determination whether or not resources are insufficient, not only the utilization rate of CPU but also a plurality of factors such as free capacity of storage, the utilization rate of memory, a network bandwidth, or the number of accesses which are concurrently accessed are involved, it is preferable to provide a reference in a combination of these factors.

Alternatively, in the case where the utilization rate of resources of a plurality of dedicated virtual machines used by one user is equal to or less than a reference value, the function providing means 27 stops use of at least one of a plurality of dedicated virtual machines of the user. For example, the resources of dedicated virtual machines are periodically measured. Since, in the case where the utilization rate of resources is equal to or less than a second reference value, there is a space margin of the resources, the use of one of the dedicated virtual machines is stopped when a plurality of dedicated virtual machines are started. The second reference value is set to be a value less than at least the first reference value. For example, in the case where the utilization rate of CPU usage is equal to or less than 20%, a sufficient processing speed is secured. Therefore, in a case where the utilization rate of CPU is equal to or less than 20%, one of the dedicated virtual machines is released, thereby stopping the use of one of the dedicated virtual machines. In the example of FIG. 3, the user group M₂ uses two dedicated virtual machines. However, since the utilization rate of resources is less than the second reference value, one dedicated virtual machine is released.

The monitoring means 28 monitors the virtual machine starting means 22, the dedicated virtual machine setting means 25, and the function providing means 27, and monitors the number of starts of common virtual machines, a change from a common virtual machine to a dedicated virtual machine, and the utilization rate of resources of a dedicated virtual machines.

In each of a plurality of common virtual machines, the connecting means 29 connects a storage unit 31, in which data used for the common application is readable or writable, to a dedicated virtual machine. Preferably, the storage unit 31 is a virtual storage unit on a cloud, and is set as a dedicated storage unit with respect to a user after a setting of a parameter is performed. The virtual storage unit indicates a storage for storing data in the cloud, and includes, for example, box (registered trademark), Dropbox (registered trademark), Onedrive (registered trademark), GoogleDrive (registered trademark), and the like. A storage region accessible in a unit of each user or user group is provided as a dedicated virtual storage unit in these virtual storage units, and the connecting means 29 performs a setting for connection to the dedicated virtual storage unit to be readable and writeable by an application on the dedicated virtual machine. Even after the dedicated virtual machine is released with respect to a virtual storage unit used by a specified user, the connecting means 29 performs a setting such that the dedicated virtual machine may be connected again to the same virtual storage unit when the dedicated virtual machine for the same user is started. As a result, the user may always use the same virtual storage unit whenever the user logs in. That is, for example, whenever the hospital M₁ accesses to the virtual storage unit, data may be stored in the same state as a state before the previous log-out. Hereinafter, the storage unit 31 is explained as a virtual storage unit 31.

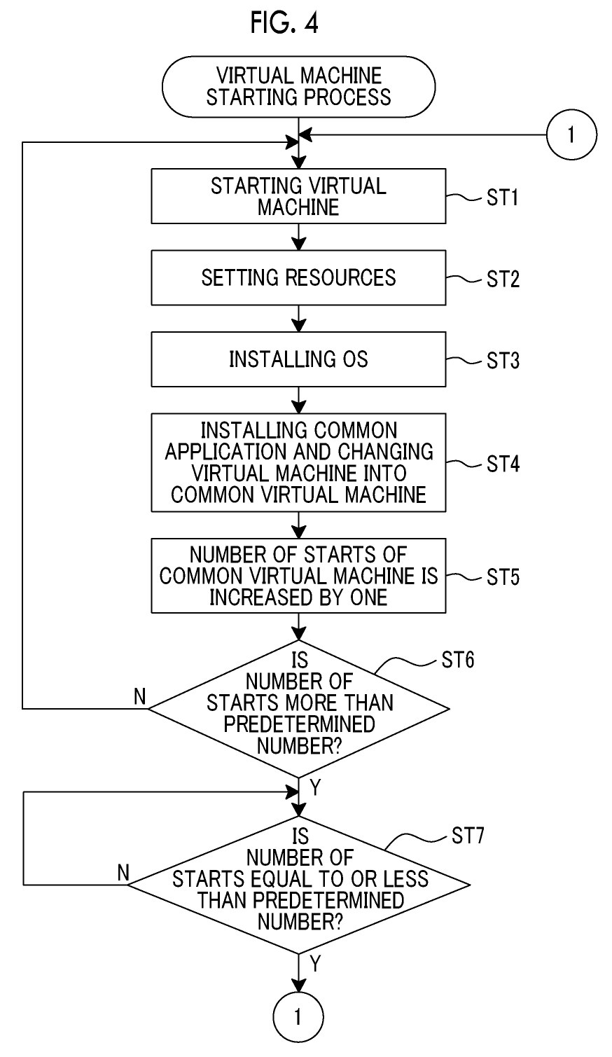

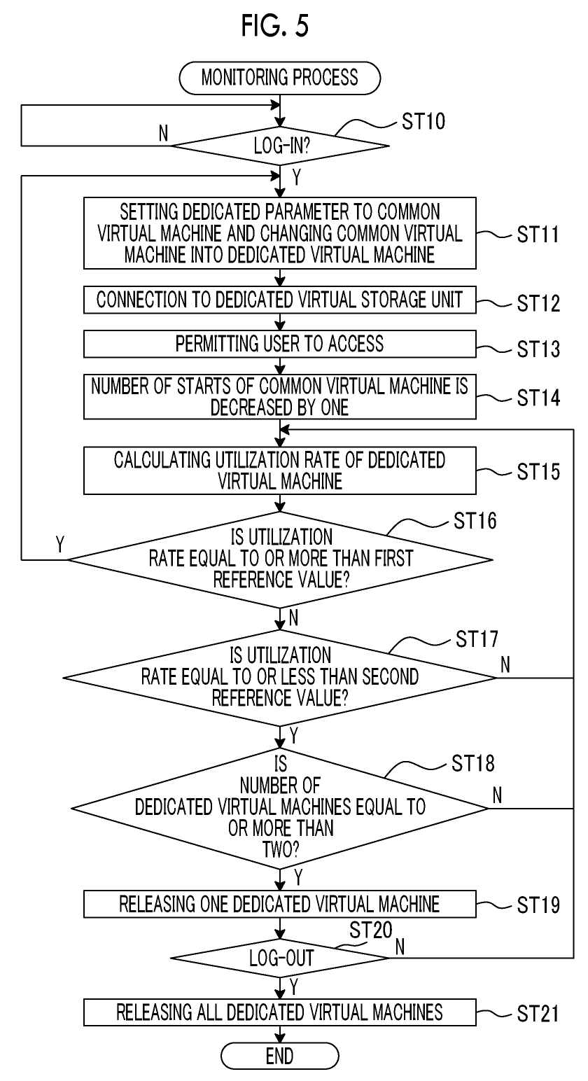

Next, according to flowcharts in FIG. 4 and FIG. 5, a flow of processing of an application providing system will be described. FIG. 4 is a flowchart showing a flow of processing for starting a common virtual machine. FIG. 5 is a flowchart showing a flow of processing for changing a started common virtual machine into a dedicated virtual machine. In the present embodiment, there is described a case where a dedicated virtual machine for each user group is created. In the present embodiment, there is described a case that processing for starting the common virtual machine and processing for changing the common virtual machine into the dedicated virtual machine are performed in parallel.

First, according to the flowchart in FIG. 4, starting of the common virtual machine is described. In the application providing server 20, the monitoring means 28 starts a virtual machine by using the virtual machine starting means 22 (step ST1) and sets a constant resource according to a defined value (step ST2). Furthermore, after installing OS on the virtual machine (step ST3), the virtual machine is changed into a common virtual machine by reading a common application from an application storage means 23 and installing the common application (step ST4). The number of starts in a counter of common virtual machines is counted up by one (step ST5). Until the number of starts of common virtual machines is more than a predetermined number, for example, ten, the determination of step ST6 is negative and the processing returns to step ST1. Furthermore, step ST1 to step ST6 are repeated again, and the processing for starting the common virtual machine is executed until the number of starts of common virtual machines is more than the predetermined number.

When the number of starts of common virtual machines is more than the predetermined number, the determination of step ST6 is affirmative, and starting of the common virtual machine is stopped for a moment. As it is, until the number of starts is equal to or less than a predetermined number, the processing is in a standby state and the determination of step ST7 is negative. Once the common virtual machine is changed into a dedicated virtual machine (as described later in detail), the number of starts of common virtual machines is decreased and the number of starts of common virtual machines becomes equal to or less than a predetermined value. Once it is detected that the number of starts of common virtual machines becomes equal to or less than the predetermined value, the determination of step ST7 is affirmative, the processing returns to step ST1, and step ST1 to step ST6 are executed to start a new common virtual machine.

Next, according to the flowchart in FIG. 5, a flow of processing for changing a started common virtual machine into a dedicated virtual machine will be described. For example, once a user of a hospital M₁ inputs an ID and logs in, the access control means 24 authorizes the ID of the user group M₁. Once the ID is authorized, the determination of step ST10 is affirmative. Subsequently, using the dedicated virtual machine setting means 25, the monitoring means 28 reads a parameter corresponding to the hospital M₁ in the common application, from the parameter storage means 26, sets the dedicated parameter of the hospital M₁ which logs in the common virtual machine, in the common application, and changes one of common virtual machines into a dedicated virtual machine of the hospital M₁ (step ST11). Specifically, a setting of an image processing parameter of the image processing corresponding to the hospital M₁ and a setting of a format of a diagnosis report of the diagnosis report creation support processing corresponding to the hospital M₁ is performed. Also, the connecting means 29 performs a setting of a parameter for connection to the dedicated virtual storage unit 31, for example, GoogleDrive, of the user group M₁ (step ST12). By this setting, data used for the application may be read from or written to the dedicated virtual storage unit 31.

Once these settings are completed, the access control means 24 permits the user to access the dedicated virtual machine (step ST13). As a result, the application from the application providing server 20 may be used in the terminal apparatus 10. For example, it is possible to transmit an image data imaged in the hospital M₁ from the terminal apparatus 10 of the hospital M₁ to the application providing server 20, execute image processing on application providing server 20, and receive a result of the image processing on the terminal apparatus 10. Furthermore, it is possible to store the image data and the result of the image processing into the dedicated virtual storage unit 31 of the user group M₁. Alternatively, it is possible to execute the diagnosis report creation support processing by the application providing server 20, create a diagnosis report according to an input from the terminal apparatus 10, and store the diagnosis report into a diagnosis report database connected to a local area network of the hospital.

In the processing of step ST11 to step ST13, since one of common virtual machines is changed into the dedicated virtual machine, the number of starts of common virtual machines is counted down by one (step ST14). Subsequently, the utilization rate of resources of the dedicated virtual machine is calculated (step ST15). In a case where the utilization rate is equal to or more than a first reference value, for example, 80%, the determination of step ST16 is negative. To prevent processing capability of the dedicated virtual machine from being degraded resulting from the insufficiency of the resources of the dedicated virtual machine of the user, the function providing means 27 returns to step ST11, to set a parameter corresponding to the user group M₁ to one of common virtual machines and add a dedicated virtual machine, using the dedicated virtual machine setting means 25. Furthermore, the connecting means 29 performs a setting for connecting the added dedicated virtual machine to the dedicated virtual storage unit 31 of the user group M₁ (step ST12). The user is permitted to access the dedicated virtual machine (step ST13). Also, the number of starts of common virtual machines is counted down by one (step ST14). As described above, once the processing capability of the dedicated virtual machine is degraded, one of common virtual machines is always changed into a dedicated virtual machine. Therefore, the processing capability is maintained at a certain level or higher.

Once a common virtual machine is changed into a dedicated virtual machine, the number of starts of common virtual machines is decreased. In the process of starting a common virtual machine shown in FIG. 4, the monitoring means 28 detects that the number of starts of common virtual machines is equal to or less than the predetermined value, the determination of step ST7 is affirmative, step ST1 to step ST6 are repeated, and a common virtual machine is started until the number of starts of common virtual machines becomes the predetermined number.

Returning to FIG. 5, there will be described a case where the utilization rate of resources of dedicated virtual machines is low. In a case where the utilization rate of resources is equal to or less than 80%, the determination of step ST16 is negative, and furthermore, it is determined whether the utilization rate of resources is equal to or less than the second reference value. For example, in a case where the utilization rate is equal to or less than 20%, it is considered that the utilization rate is low and resources are secured more than necessary. In the case where the utilization rate of resources is 20% to 80%, it is considered that proper dedicated virtual machines are started. For example, in a case where the utilization rate of resources is 40%, the determination of step ST17 is negative, and step ST15 to step ST17 are repeated to monitor the utilization rate of resources until the utilization rate of resources is measured again and the utilization rate of resources is changed.

In the case where the utilization rate of resources is equal to or less than 20% and the number of dedicated virtual machines of the user group M₁ is two or more (determinations of step ST17 and step ST18 are affirmative), since resources are secured more than necessary, the function providing means 27 releases one of the dedicated virtual machines of the user group M₁. Until the user logs out, the determination of step ST20 is negative, and the processing returns to step ST15 and the utilization rate of resources is measured again. As described above, while the utilization rate of resources of dedicated virtual machines is calculated until the user logs out, the processing of changing a common virtual machine into a dedicated virtual machine and the processing of releasing the dedicated virtual machine are repeated so that the resources of dedicated virtual machines used by the user may be secured within a proper range.

Once the user logs out, the access control means 24 releases all the dedicated virtual machines. In this way, once the user logs out, all the dedicated virtual machines are released and prevented from being reused, so that there is no reuse of a common virtual machine used by another user. Accordingly, since no data used by another user remains in the dedicated virtual machine, it is possible to enhance confidentiality.

The case of log-in using one account is described above. However, a user may log in with a plurality of accounts in a user group, or the dedicated virtual machine may be allocated in a unit of a user group. Also, in this case, the fact that all the dedicated virtual machines are released means that all the users of the user group log out.

As described above in detail, by requiring a common virtual machine beforehand and changing the common virtual machine into a dedicated virtual machine as necessary, it is possible to change the allocation of resources without taking time, and it is possible to always provide a user with a pleasant execution environment of the application.

In the embodiments, hardware structures of the processing unit to execute various kinds of processes of the application providing server means various kinds of processors shown as below. The various kinds of processors include a Central Processing Unit (CPU) that is a general-purpose processor for executing software (program) and functioning as various processing units, a Programmable Logic Device (PLD) such as a Field Programmable Gate Array (FPGA), which is a processor having a circuit configuration changeable after manufacturing, or a dedicated electric circuit such as an Application Specific Integrated Circuit (ASIC), which is a processor having a circuit configuration dedicatedly designed to execute specified processing, and so on.

One processing unit may be configured with one of the various kinds of processors, or in a combination of homogeneous or heterogeneous two or more processors (for example, a plurality of FPGAs, or a combination of a CPU and an FPGA). Also, a plurality of processing units may be configured with one processor. An example of configuring a plurality of processing units by means of one processor includes a first form in which one processor is configured in a combination of one or more CPUs and software, and this processor functions as a plurality of processing units, as represented by a computer of a client and server, and the like. There is a second form in which a processor is used for implementing all the functions of a system including a plurality of processing units on one Integrated Circuit (IC) chip, as represented by a System On Chip (SoC), and the like. In this way, various kinds of processing units are configured with one or more various kinds of processors as the hardware structures.

What is claimed is:

1. An application providing apparatus comprising:
virtual machine starting means for starting each of a plurality of common virtual machines in which a common application program is set;
dedicated virtual machine setting means for setting the common virtual machine to which a parameter for any user of a plurality of users is set, as a dedicated virtual machine of the user for which a setting of the parameter is performed, in the common application program set in each of the plurality of common virtual machines; and
function providing means for providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.

2. The application providing apparatus according to claim 1,
wherein, in a case where a utilization rate of resources of a plurality of dedicated virtual machines used by one user is equal to or less than a reference value, the function providing means stops use of at least one of the plurality of dedicated virtual machines of the one user.

3. The application providing apparatus according to claim 1,
wherein each of the plurality of common virtual machines is a virtual machine to which a constant resource is allocated.

4. The application providing apparatus according to claim 1,
wherein each of the plurality of users is a hospital,
the common application program includes a program for performing image processing or a program for performing diagnosis report creation support processing, and
the setting of the parameter is a setting of an image processing parameter of the image processing corresponding to the hospital, in a case of the program for performing the image processing, or a setting of a format of a diagnosis report of the diagnosis report creation support processing corresponding to the hospital, in a case of the program for performing the diagnosis report creation support processing.

5. The application providing apparatus according to claim 1,
wherein each of the plurality of common virtual machines has a storage unit in which data used for the common application program is readable or writable, and
a setting of the parameter allows the storage unit to be set as a dedicated storage unit with respect to a user for which the setting of the parameter is performed.

6. An application providing method, comprising:
a virtual machine starting step of starting each of a plurality of common virtual machines in which a common application program is set;
a dedicated virtual machine setting step of setting the common virtual machine to which a parameter for any user of a plurality of users is set, as a dedicated virtual machine of the user for which a setting of the parameter is performed, in the common application program set in each of the plurality of common virtual machines; and
a function providing step of providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient, which are performed by an application providing apparatus.

7. A non-transitory computer-readable recording medium storing therein an application providing program that causes a computer to perform:
a virtual machine starting step of starting each of a plurality of common virtual machines in which a common application program is set;
a dedicated virtual machine setting step of setting the common virtual machine to which a parameter for any user of a plurality of users is set, as a dedicated virtual machine of the user for which a setting of the parameter is performed, in the common application program set in each of the plurality of common virtual machines; and
a function providing step of providing a function by additionally setting at least one of the common virtual machines for which a setting of the parameter is not performed, as another dedicated virtual machine of a user corresponding to a dedicated virtual machine of which resources are insufficient, in a case where resources of the dedicated virtual machine are insufficient.
